(19) United States
(12) Patent Application Publication
(10) Pub. No.: US 2026/0358215 A1
(43) Pub. Date: Nov. 12, 2026

(54) ODOR IDENTIFICATION METHOD AND ODOR IDENTIFICATION SYSTEM

(71) Applicant: PANASONIC HOUSING SOLUTIONS CO., LTD., Osaka (JP)

(72) Inventor: Toshiki NIINOMI, Miyagi (JP)

(21) Appl. No.: 19/121,766

(22) PCT Filed: Oct. 4, 2023
(86) PCT No.: PCT/JP2023/036173

§ 371 Date: Apr. 16, 2025

(30) Foreign Application Priority Data
Oct. 28, 2022 (JP) 2022-173103

(51) Int. Cl. G01N 5/02; G01N 27/12

(52) U.S. Cl. CPC G01N 5/02; G01N 27/12

(57) ABSTRACT

An odor identification method includes: (a) causing a first sensor, a second sensor, and a third sensor to adsorb an odorant contained in sample gas at different times in a first period, and to desorb the odorant at different times in a second period; (b) measuring respective detection signals output from the first sensor, the second sensor, and the third sensor in a common measurement period (ΔTs) that is shorter than the first period; and (c) identifying the odor of the sample gas, based on the respective detection signals measured.

### TECHNICAL FIELD

The present disclosure relates to an odor identification method and an odor identification system.

### BACKGROUND ART

An odor identification method is known which extracts the feature of the signal output from a sensor exposed to a sample gas, and identifies the odor of the sample gas, based on the feature. As an example of this odor identification method, Patent Literature (PTL) 1 discloses identifying an analyte, using, as the feature, the intensity, the wavelength, the intensity ratio, and the kurtosis of the pulsed signal used for detecting the analyte.

### CITATION LIST

### Patent Literature

[PTL 1] International Publication No. WO 2018/207524

### SUMMARY OF INVENTION

### Technical Problems

The typical odor identification method requires however a relatively long time to measure (or collect) data necessary for identifying the odor of the sample gas.

To address the problem, the present disclosure provides an odor identification method and an odor identification system that enables identification of the odor of a sample gas in a short time and with high accuracy.

### Solution to Problems

An odor identification method according to an aspect of the present disclosure is a method of identifying an odor of a sample gas. The odor identification method includes: (a) causing a plurality of sensors to adsorb an odorant contained in the sample gas at different times in a first period, and to desorb the odorant at different times in a second period, the plurality of sensors outputting respective detection signals according to an adsorption concentration of the odorant contained in the sample gas; (b) measuring the respective detection signals output from the plurality of sensors in a common measurement period that is shorter than the first period or the second period; and (c) identifying the odor of the sample gas, based on the respective detection signals measured in the (b) measuring.

An odor identification system according to an aspect of the present disclosure is for identifying an odor of a sample gas. The odor identification system includes: a plurality of sensors that output respective detection signals according to an adsorption concentration of an odorant contained in the sample gas; a measurer that measures the respective detection signals output from the plurality of sensors in a common measurement period that is shorter than a first period or a second period, when the plurality of sensors adsorb the odorant contained in the sample gas at different times in the first period, and desorb the odorant contained in the sample gas at different times in the second period; and an identifier that identifies the odor of the sample gas, based on the respective detection signals measured by the measurer.

Note that these general and specific aspects of the present disclosure may be implemented using a system, a method, an integrated circuit, a computer program, or a computer-readable recording medium, such as a CD-ROM, or any combination of systems, methods, integrated circuits, computer programs, or recording media.

### Advantageous Effects of Invention

An odor identification method, and so on, according to an aspect of the present disclosure enables identification of the odor of a sample gas in a short time and with high accuracy.

### BRIEF DESCRIPTION OF DRAWINGS

FIG. 1 is a block diagram showing a configuration of an odor identification system according to Embodiment 1.

FIG. 2 is a schematic view showing an exposer of the odor identification system according to Embodiment 1.

FIG. 3 shows a first sensor of the odor identification system according to Embodiment 1.

FIG. 4A is a graph showing example responses of sensing elements, when the first sensor is exposed to sample gas A.

FIG. 4B is a graph showing example responses of the sensing elements, when a second sensor is exposed to sample gas B.

FIG. 4C is a graph showing example responses of the sensing elements, when a third sensor is exposed to sample gas C.

FIG. 5 is a graph schematically showing respective detection signals output from the first sensor, the second sensor, and the third sensor.

FIG. 6 illustrates an example of measurement processing by a measurer of the odor identification system according to Embodiment 1.

FIG. 7 illustrates another example of the measurement processing by the measurer of the odor identification system according to Embodiment 1.

FIG. 8 illustrates an example of identification processing by an identifier of the odor identification system according to Embodiment

FIG. 9 is a flowchart showing the flow of an operation of the odor identification system according to Embodiment 1.

FIG. 10 illustrates a configuration of an odor identification system according to Comparative Example A.

FIG. 11 illustrates a configuration of an odor identification system according to Comparative Example B.

FIG. 12 is a table showing a result of Experiment 1 for confirming the advantages of the odor identification system according to Embodiment 1.

FIG. 13 is a table showing a result of Experiment 2 for confirming the advantages of the odor identification system according to Embodiment 1.

FIG. 14 is a block diagram showing a configuration of an odor identification system according to Embodiment 2.

FIG. 15 is a schematic view showing an exposer of the odor identification system according to Embodiment 2.

FIG. 16 is a graph schematically showing respective detection signals output from a first sensor, a second sensor, and a third sensor.

### DESCRIPTION OF EMBODIMENTS

### Technique 1

Technique 1 is directed to an odor identification method of identifying an odor of a sample gas. The odor identification method includes: (a) causing a plurality of sensors to adsorb an odorant contained in the sample gas at different times in a first period, and to desorb the odorant at different times in a second period, the plurality of sensors outputting respective detection signals according to an adsorption concentration of the odorant contained in the sample gas; (b) measuring the respective detection signals output from the plurality of sensors in a common measurement period that is shorter than the first period or the second period; and (c) identifying the odor of the sample gas, based on the respective detection signals measured in the (b) measuring.

According to Technique 1, in the common measurement period, which is shorter than the first period or the second period, the respective detection signals output from the plurality of sensors are measured. Based on the respective detection signals measured, the odor of the sample gas is identified based on the respective detection signals measured. Since the common measurement period for measuring the respective detection signals is shorter than the first period or the second period, the time required for the measurement can be shortened, and the odor of the sample gas can be identified in the short time. In addition, since the detection signals are measured in the common measurement period, a larger amount of information can be obtained through the measurement. As a result, the odor of the sample gas can be identified with high accuracy.

### Technique 2

Technique 2 is directed to the odor identification method according to an embodiment of Technique 1. The plurality of sensors are contained in respective containers. A plurality of selector valves are provided in association with the respective containers, the plurality of selector valves each being switched between a first state and a second state, the sample gas being introduced into the respective containers in the first state, the sample gas is discharged from the respective containers in the second state. The (a) causing includes switching the plurality of selector valves from one of the first state or the second state to another at different times.

According to Technique 2, the plurality of selector valves are controlled to be easily switched from one of the first state or the second state to the other at different times.

### Technique 3

Technique 3 is directed to the odor identification method according to an embodiment of Technique 1. A plurality of temperature control devices are provided in association with the plurality of sensors that alternate a heating mode and a non-heating mode, the plurality of sensors being heated in the heating mode, the plurality of sensors being not heated in the non-heating mode. The (a) causing includes switching the plurality of temperature control devices from one of the heating mode or the non-heating mode to another of the heating mode or the non-heating at different times.

According to Technique 3, the plurality of temperature control devices can be controlled to be easily switched from one of the heating mode or the non-heating mode to the other at different times.

### Technique 4

Technique 4 is directed to the odor identification method according to an embodiment of any one of Techniques 1 to 3. The the (a) causing includes: causing the plurality of sensors to adsorb the odorant at different times in the first period to cause the respective detection signals output from the plurality of sensors to have a predetermined phase difference; and causing the plurality of sensors to desorb the odorant at different times in the second period to cause the respective detection signals output from the plurality of sensors to have the predetermined phase difference.

According to Technique 4, in the first period or the second period, the respective detection signals output from the plurality of sensors have the predetermined phase difference. A plurality of partial detection signals provide the amount of information corresponding to the detection signals output during the first period or the second period.

### Technique 5

Technique 5 is directed to the odor identification method according to an embodiment of Technique 4. The predetermined phase difference is obtained by dividing the first period or the second period by a total number of the plurality of sensors.

According to Technique 5, a plurality of partial detection signals effectively provide the amount of information corresponding to the detection signals output during the first period or the second period.

### Technique 6

Technique 6 is directed to the odor identification method according to an embodiment of any one of Techniques 1 to 5. The (c) identifying includes generating a pseudo detection signal by connecting waveforms of the respective detection signals measured in the (b) measuring, and identifying the odor of the sample gas based on the pseudo detection signal generated, using a trained model for identifying the sample gas.

Technique 6 enables identification of the odor of the sample gas with high accuracy.

### Technique 7

Technique 7 is directed to the odor identification method according to an embodiment of any one of Techniques 1 to 5. The (c) identifying includes calculating respective features of the respective detection signals measured in the (b) measuring, and identifying the odor of the sample gas based on the respective features calculated, using a trained model for identifying the sample gas.

Technique 7 enables identification of the odor of the sample gas with high accuracy.

### Technique 8

Technique 8 is directed to the odor identification method according to an embodiment of any one of Techniques 1 to 7. The odor identification method further includes: (d) obtaining, via a network, the respective detection signals output from the plurality of sensors in the first period or the second period.

Technique 8 easily provides the respective detection signals output from the plurality of sensors even at remote locations via the network.

### Technique 9

Technique 9 is directed to the odor identification method according to an embodiment of any one of Techniques 1 to 8. Each of the plurality of sensors includes a plurality of sensing elements with different sensing characteristics.

According to Technique 9, the plurality of sensing elements output respective detection signals in one sensor, which enables identification of the odor of the sample gas with high accuracy.

### Technique 10

Technique 10 is directed to an odor identification system for identifying an odor of a sample gas. The odor identification system includes: a plurality of sensors that output respective detection signals according to an adsorption concentration of an odorant contained in the sample gas; a measurer that measures the respective detection signals output from the plurality of sensors in a common measurement period that is shorter than a first period or a second period, when the plurality of sensors adsorb the odorant contained in the sample gas at different times in the first period, and desorb the odorant contained in the sample gas at different times in the second period; and an identifier that identifies the odor of the sample gas, based on the respective detection signals measured by the measurer.

According to Technique 10, the measurer measures the respective detection signals output from the plurality of sensors in the common measurement period, which is shorter than the first period or the second period. The identifier identifies the odor of the sample gas, based on the respective detection signals measured. Since the common measurement period for measuring the respective detection signals is shorter than the first period or the second period, the time required for the measurement can be shortened, and the odor of the sample gas can be identified in the short time. In addition, since the detection signals are measured in the common measurement period, a larger amount of information can be obtained through the measurement. As a result, the odor of the sample gas can be identified with high accuracy.

Note that these general and specific aspects of the present disclosure may be implemented using a system, a method, an integrated circuit, a computer program, or a computer-readable recording medium, such as a CD-ROM, or any combination of systems, methods, integrated circuits, computer programs, or recording media.

Now, embodiments will be described in detail with reference to the drawings.

The embodiments described below are mere general or specific examples of the present disclosure. The numerical values, shapes, materials, elements, the arrangement and connection of the elements, steps, step orders etc. shown in the following embodiments are thus mere examples, and are not intended to limit the scope of the present disclosure. Among the elements in the following embodiments, those not recited in the independent claims, which define the broadest concept, will be described as optional.

### Embodiment 1

### 1-1. Configuration of Odor Identification System

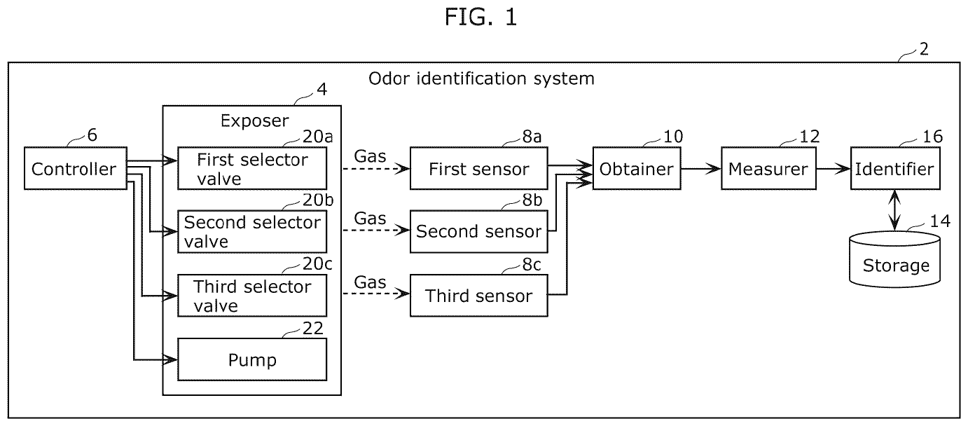

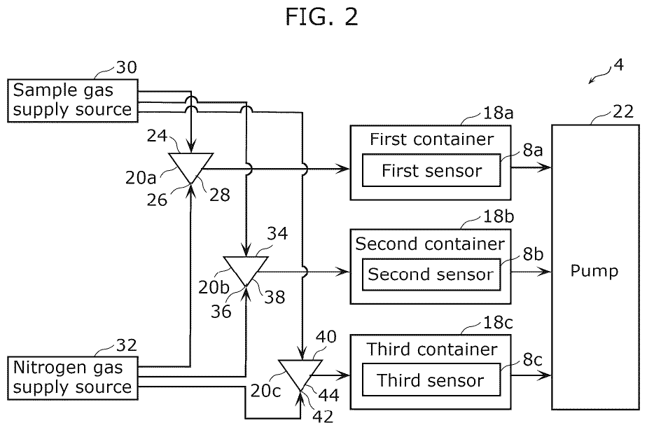

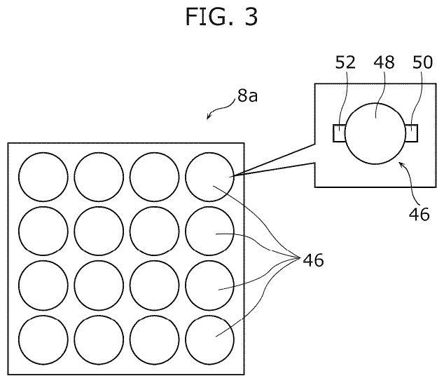

A configuration of odor identification system 2 according to Embodiment 1 will be described with reference to FIGS. 1 to 3. FIG. 1 is a block diagram showing the configuration of odor identification system 2 according to Embodiment 1. FIG. 2 is a schematic view showing exposer 4 of odor identification system 2 according to Embodiment 1. FIG. 3 shows first sensor 8a of odor identification system 2 according to Embodiment 1.

Odor identification system 2 is for identifying the odor of a sample gas. Specifically, odor identification system 2 identifies which kind of odorant is contained in the sample gas. For example, the sample gas is collected from food, and odor identification system 2 is applied to full inspection as to whether the food transported one by one on a conveyor in a food production line causes gas with an unusual odor.

As shown in FIG. 1, odor identification system 2 includes exposer 4, controller 6, first sensor 8a (as an example of the sensor), second sensor 8b (as another example of the sensor), third sensor 8c (as further another example of the sensor), obtainer 10, measurer 12, storage 14, and identifier 16.

Exposer 4 is a mechanism for exposing first sensor 8a, second sensor 8b, and third sensor 8c to a sample gas or nitrogen gas. Note that the nitrogen gas is inert gas containing no odorant and used for removing the odorant from first sensor 8a, second sensor 8b, and third sensor 8c. The nitrogen gas may be other inert gas, such as air, or gas obtained by removing chemicals from the sample gas using a filter or any other suitable means may be used.

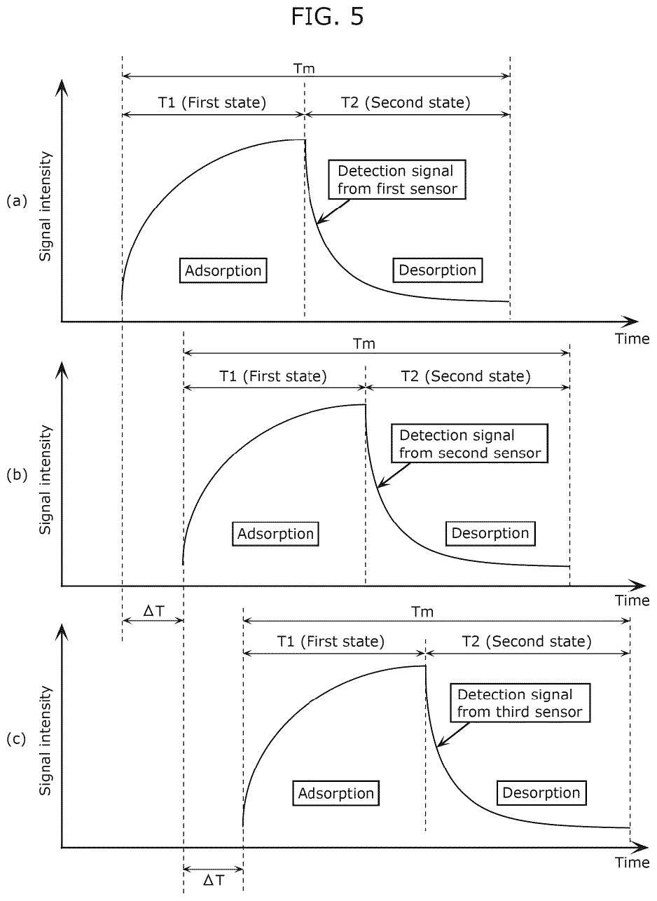

Specifically, exposer 4 exposes first sensor 8a to the sample gas in first period T1 (e.g., six seconds) out of period Tm (see FIG. 5 which will be described later). Period Tm includes first period T1 and second period T2 (e.g., 18 seconds) following first period T1. Exposer 4 further exposes first sensor 8a to the nitrogen gas in second period T2 out of period Tm.

Exposer 4 further exposes second sensor 8b to the sample gas in first period T1 out of period Tm with a short delay from the start of exposing first sensor 8a to the sample gas. Exposer 4 further exposes second sensor 8b to the nitrogen gas in second period T2 out of period Tm with a short delay from the start of exposing first sensor 8a to the nitrogen gas.

Exposer 4 further exposes third sensor 8c to the sample gas in first period T1 out of period Tm with a short delay from the start of exposing second sensor 8b to the sample gas. Exposer 4 further exposes third sensor 8c to the nitrogen gas in second period T2 out of period Tm with a short delay from the start of exposing second sensor 8b to the nitrogen gas.

Here, a specific configuration of exposer 4 will be described with reference to FIG. 2. As shown in FIG. 2, exposer 4 includes first container 18a (as an example of the container), second container 18b (as another example of the container), third container 18c (as further another example of the container), first selector valve 20a (as an example of the selector valve), second selector valve 20b (as another example of the selector valve), third selector valve 20c (as further another example of the selector valve), and pump 22.

First container 18a is, for example, a box-shaped container for containing first sensor 8a. Second container 18b is, for example, a box-shaped container for containing second sensor 8b. Third container 18c is, for example, a box-shaped container for containing third sensor 8c. As will be described later, the sample gas or the nitrogen gas is introduced into first container 18a, second container 18b, and third container 18c.

First selector valve 20a is a three-way electromagnetic valve for switching the gas to be introduced into first container 18a and is driven by controller 6 (see FIG. 1). First selector valve 20a includes first inlet port 24, second inlet port 26, and outlet port 28. First selector valve 20a can be switched between first and second states. In the first state, first inlet port 24 and outlet port 28 communicate with each other. In the second state, second inlet port 26 and outlet port 28 communicate with each other. In the first state, first inlet port 24 and outlet port 28 are open, and second inlet port 26 is closed. On the other hand, in the second state, second inlet port 26 and outlet port 28 are open, and first inlet port 24 is closed.

First inlet port 24 communicates, via a pipe, with sample gas supply source 30 for supplying the sample gas. Second inlet port 26 communicates, via a pipe, with nitrogen gas supply source 32 for supplying the nitrogen gas. Outlet port 28 communicates with first container 18a via a pipe.

Second selector valve 20b is a three-way electromagnetic valve for switching the gas to be introduced into second container 18b and is driven by controller 6. Second selector valve 20b includes first inlet port 34, second inlet port 36, and outlet port 38. Second selector valve 20b is switched between first and second states. In the first state, first inlet port 34 and outlet port 38 communicate with each other. In the second state, second inlet port 36 and outlet port 38 communicate with each other. In the first state, first inlet port 34 and outlet port 38 are open, and second inlet port 36 is closed. On the other hand, in the second state, second inlet port 36 and outlet port 38 are open, and first inlet port 34 is closed.

First inlet port 34 communicates with sample gas supply source 30 via a pipe. Second inlet port 36 communicates with nitrogen gas supply source 32 via a pipe. Outlet port 38 communicates with second container 18b via a pipe.

Third selector valve 20c is a three-way electromagnetic valve for switching the gas to be introduced into third container 18c and is driven by controller 6. Third selector valve 20c includes first inlet port 40, second inlet port 42, and outlet port 44. Third selector valve 20c is switched between first and second states. In the first state, first inlet port 40 and outlet port 44 communicate with each other. In the second state, second inlet port 42 and outlet port 44 communicate with each other. In the first state, first inlet port 40 and outlet port 44 are open, and second inlet port 42 is closed. On the other hand, in the second state, second inlet port 42 and outlet port 44 are open, and first inlet port 40 is closed.

First inlet port 40 communicates with sample gas supply source 30 via a pipe. Second inlet port 42 communicates with nitrogen gas supply source 32 via a pipe. Outlet port 44 communicates with third container 18c via a pipe.

Pump 22 is a suction pump driven by controller 6. Pump 22 communicates with first container 18a, second container 18b, and third container 18c via pipes, and also communicates with an exhaust duct (not shown) via a pipe. Pump 22 introduces the sample gas or the nitrogen gas into first container 18a, and discharges the sample gas or the nitrogen gas introduced into first container 18a to an exhaust duct. Pump 22 also introduces the sample gas or the nitrogen gas into second container 18b, and discharges the sample gas or the nitrogen gas introduced into second container 18b to the exhaust duct. Pump 22 also introduces the sample gas or the nitrogen gas into third container 18c, and discharges the sample gas or the nitrogen gas introduced into third container 18c to the exhaust duct.

The sample gas or the nitrogen gas is introduced into first container 18a as follows. In first period T1 of period Tm, with pump 22 driven, first selector valve 20a is switched to the first state. In this first state, the sample gas supplied from sample gas supply source 30 is introduced via first inlet port 24 and outlet port 28 of first selector valve 20a into first container 18a. Accordingly, first sensor 8a is exposed to the sample gas introduced into first container 18a, and the odorant contained in the sample gas is adsorbed by first sensor 8a. The sample gas introduced into first container 18a is also discharged to the exhaust duct via pump 22.

In second period T2 of period Tm, with pump 22 driven, first selector valve 20a is switched to the second state. In this second state, the nitrogen gas supplied from nitrogen gas supply source 32 is introduced via second inlet port 26 and outlet port 28 of first selector valve 20a into first container 18a. At this time, the sample gas remaining inside first container 18a is discharged to the exhaust duct via pump 22 by the flow of the nitrogen gas introduced into first container 18a. Accordingly, first sensor 8a is exposed to the nitrogen gas introduced into first container 18a, and the odorant adsorbed by first sensor 8a is desorbed due to the flow of the nitrogen gas. The nitrogen gas introduced into first container 18a is discharged to the exhaust duct via pump 22.

The sample gas or the nitrogen gas is introduced into second container 18b as follows. In first period T1 of period Tm, with pump 22 driven, second selector valve 20b is switched to the first state. In this first state, the sample gas supplied from sample gas supply source 30 is introduced via first inlet port 34 and outlet port 38 of second selector valve 20b into second container 18b. Accordingly, second sensor 8b is exposed to the sample gas introduced into second container 18b, and the odorant contained in the sample gas is adsorbed by second sensor 8b. The sample gas introduced into second container 18b is discharged to the exhaust duct via pump 22.

In second period T2 of period Tm, with pump 22 driven, second selector valve 20b is switched to the second state. In this second state, the nitrogen gas supplied from nitrogen gas supply source 32 is introduced via second inlet port 36 and outlet port 38 of second selector valve 20b into second container 18b. Accordingly, second sensor 8b is exposed to the nitrogen gas introduced into second container 18b, and the odorant adsorbed by second sensor 8b is desorbed due to the flow of the nitrogen gas. The nitrogen gas introduced into second container 18b is discharged to the exhaust duct via pump 22.

The sample gas or the nitrogen gas is introduced into third container 18c as follows. In first period T1 of period Tm, with pump 22 driven, third selector valve 20c is switched to the first state. In this first state, the sample gas supplied from sample gas supply source 30 is introduced via first inlet port 40 and outlet port 44 of third selector valve 20c into third container 18c. Accordingly, third sensor 8c is exposed to the sample gas introduced into third container 18c, and the odorant contained in the sample gas is adsorbed by third sensor 8c. The sample gas introduced into third container 18c is discharged to the exhaust duct via pump 22.

In second period T2 of period Tm, with pump 22 driven, third selector valve 20c is switched to the second state. In this second state, the nitrogen gas supplied from nitrogen gas supply source 32 is introduced via second inlet port 42 and outlet port 44 of third selector valve 20c into third container 18c. Accordingly, third sensor 8c is exposed to the nitrogen gas introduced into third container 18c, and the odorant adsorbed by third sensor 8c is desorbed due to the flow of the nitrogen gas. The nitrogen gas introduced into third container 18c is discharged to the exhaust duct via pump 22.

Referring back to FIG. 1, controller 6 controls the driving of first selector valve 20a and pump 22 of exposer 4. Specifically, in first period T1 of period Tm, controller 6 drives pump 22 and switches first selector valve 20a to the first state. In second period T2 of period Tm, controller 6 drives pump 22 and switches first selector valve 20a to the second state. Controller 6 then alternately switches first selector valve 20a between the first state and the second state in period Tm as a cycle.

Controller 6 also controls the driving of second selector valve 20b and pump 22 of exposer 4. Specifically, in first period T1 of period Tm with a short delay from the switching of first selector valve 20a to the first state, controller 6 drives pump 22 and switches second selector valve 20b to the first state. In second period T2 of period Tm with a short delay from the switching of first selector valve 20a to the second state, controller 6 drives pump 22 and switches second selector valve 20b to the second state. Controller 6 then alternately switches second selector valve 20b between the first state and the second state in period Tm as a cycle.

Controller 6 also controls the driving of third selector valve 20c and pump 22 of exposer 4. Specifically, in first period T1 of period Tm with a short delay from the switching of second selector valve 20b to the first state, controller 6 drives pump 22 and switches third selector valve 20c to the first state. In third period T3 of period Tm with a short delay from the switching of second selector valve 20b to the second state, controller 6 drives pump 22 and switches third selector valve 20c to the second state. Controller 6 then alternately switches third selector valve 20c between the first state and the second state in period Tm as a cycle.

Controller 6 controls the driving of first selector valve 20a, second selector valve 20b, and third selector valve 20c so as to switch first selector valve 20a, second selector valve 20b, and third selector valve 20c from the second state to the first state at different times. Specifically, second selector valve 20b is switched from the second state to the first state with a short delay from when first selector valve 20a is switched from the second state to the first state. Third selector valve 20c is switched from the second state to the first state with a short delay from when second selector valve 20b is switched from the second state to the first state. Accordingly, first sensor 8a, second sensor 8b, and third sensor 8c adsorb the odorant contained in the sample gas at different times.

Controller 6 also controls the driving of first selector valve 20a, second selector valve 20b, and third selector valve 20c so as to switch first selector valve 20a, second selector valve 20b, and third selector valve 20c from the first state to the second state at different times. Specifically, second selector valve 20b is switched from the first state to the second state with a short delay from when first selector valve 20a is switched from the first state to the second state. Third selector valve 20c is switched from the first state to the second state with a short delay from when second selector valve 20b is switched from the first state to the second state. Accordingly, the odorants adsorbed by first sensor 8a, second sensor 8b, and third sensor 8c are desorbed due to the flow of the nitrogen gas.

First sensor 8a is contained in first container 18a so as to be exposed to the sample gas introduced into first container 18a, thereby outputting a detection signal according to the adsorption concentration of the odorant contained in the sample gas. As shown in FIG. 3, first sensor 8a is an electrically resistant sensor, for example. First sensor 8a includes a plurality of (e.g., 16) sensing elements 46 (CH1 to CH16) with different sensing characteristics. Each of the plurality of sensing elements 46 includes sensing section 48 made of a sensing film, and a pair of electrodes 50 and 52 electrically connected to sensing section 48. The electrical resistance value of sensing section 48 varies in accordance with the adsorption concentration of the odorant of the sample gas adsorbed in sensing section 48. Each of the plurality of sensing elements 46 outputs a detection signal according to the electrical resistance value of sensing section 48, as a voltage signal or a current signal, via the pair of electrodes 50 and 52 to obtainer 10.

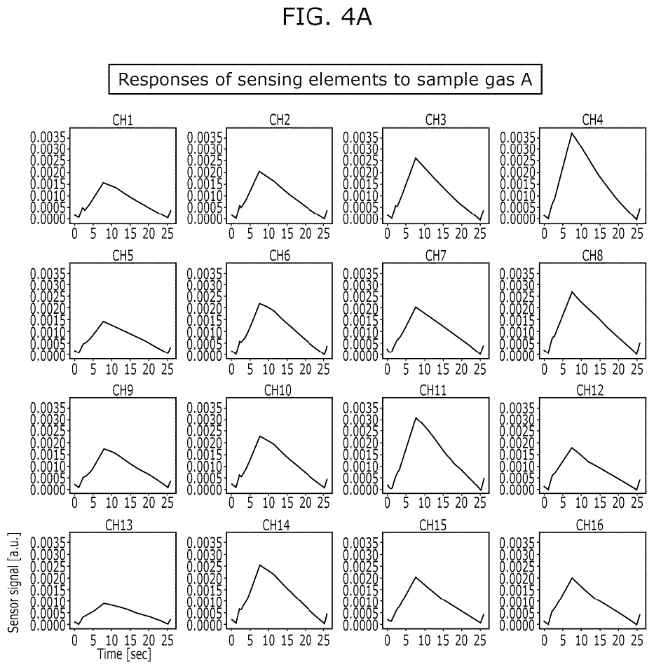

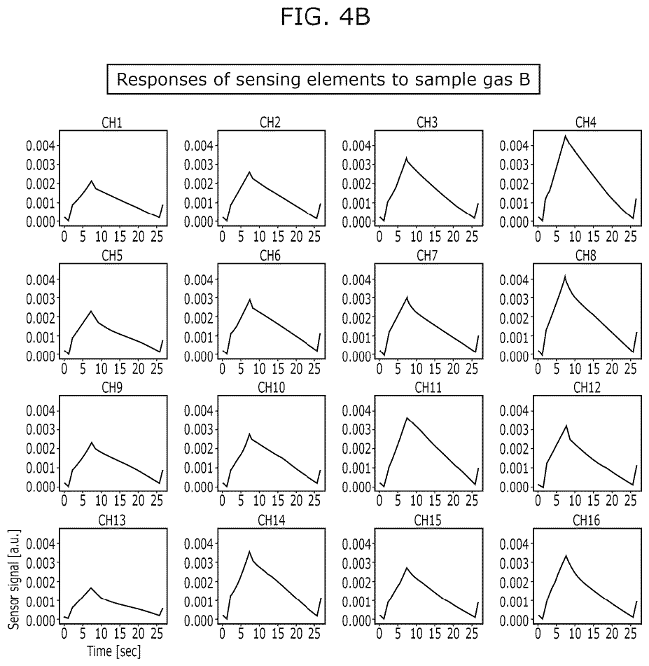

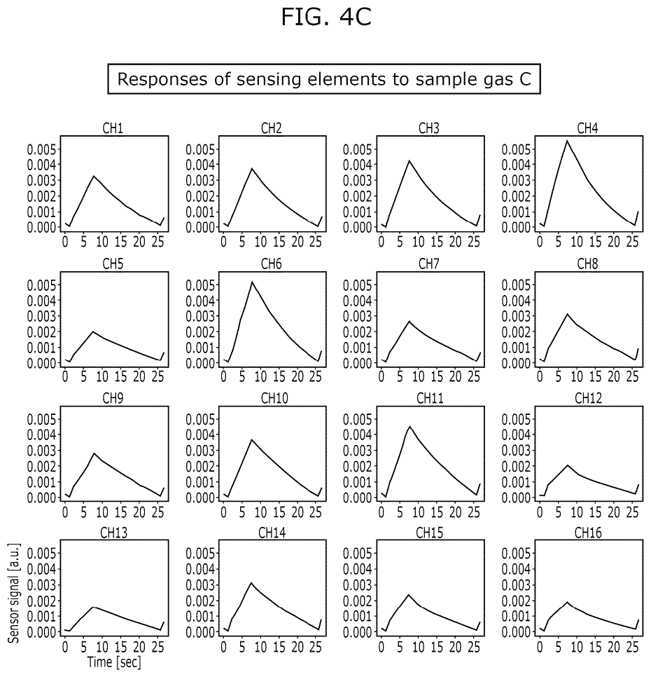

FIG. 4A is a graph showing example responses of sensing elements 46 where first sensor 8a is exposed to sample gas A (β-phenylethyl alcohol). The 16 graphs in FIG. 4A show time changes of 16 detection signals output from 16 sensing elements 46 of first sensor 8a. In each graph, the horizontal axis represents time, and the vertical axis represents the signal intensity. As shown in FIG. 4A, the electrical resistance values of 16 sensing elements 46 change with time differently from each other, where first sensor 8a is exposed to sample gas A.

FIG. 4B is a graph showing example responses of sensing elements 46 where second sensor 8b is exposed to sample gas B (methyl cyclopentenolone). The 16 graphs in FIG. 4B show time changes of 16 detection signals output from 16 sensing elements 46 of second sensor 8b. In each graph, the horizontal axis represents time, and the vertical axis represents the signal intensity. As shown in FIG. 4B, the electrical resistance values of 16 sensing elements 46 change with time differently from each other.

FIG. 4C is a graph showing example responses of sensing elements 46 where third sensor 8c is exposed to sample gas C (an isovaleric acid). The 16 graphs in FIG. 4C show time changes of 16 detection signals output from 16 sensing elements 46 of third sensor 8c. In each graph, the horizontal axis represents time, and the vertical axis represents the signal intensity. As shown in FIG. 4C, the electrical resistance values of 16 sensing elements 46 change with time differently from each other.

Second sensor 8b is contained in second container 18b so as to be exposed to the sample gas introduced into second container 18b, thereby outputting a detection signal according to the adsorption concentration of the odorant contained in the sample gas. Second sensor 8b is an electrically resistant sensor, for example. Although not shown, like first sensor 8a, second sensor 8b includes a plurality of (e.g., 16) sensing elements (CH1 to CH16) with different sensing characteristics. Each of the plurality of sensing elements includes a sensing section made of a sensing film, and a pair of electrodes electrically connected to the sensing section. The electrical resistance value of the sensing section varies in accordance with the adsorption concentration of the odorant of the sample gas adsorbed in the sensing section. Each of the plurality of sensing elements outputs a detection signal according to the electrical resistance value of the sensing section, as a voltage signal or a current signal, via the pair of electrodes to obtainer 10.

Third sensor 8c is contained in third container 18c so as to be exposed to the sample gas introduced into third container 18c, thereby outputting a detection signal according to the adsorption concentration of the odorant contained in the sample gas. Third sensor 8c is an electrically resistant sensor, for example. Although not shown, like first sensor 8a, third sensor 8c includes a plurality of (e.g., 16) sensing elements (CH1 to CH16) with different sensing characteristics. Each of the plurality of sensing elements includes a sensing section made of a sensing film, and a pair of electrodes electrically connected to the sensing section. The electrical resistance value of the sensing section varies in accordance with the adsorption concentration of the odorant of the sample gas adsorbed in the sensing section. Each of the plurality of sensing elements outputs a detection signal according to the electrical resistance value of the sensing section, as a voltage signal or a current signal, via the pair of electrodes to obtainer 10.

In FIG. 5, (a) to (c) are graphs schematically showing detection signals output from first sensor 8a, second sensor 8b, and third sensor 8c, respectively. In each graph, the horizontal axis represents time, and the vertical axis represents the signal intensity. Note that the detection signal shown in (a) of FIG. 5 is output from one of sensing elements 46 (e.g., sensing element 46-CH1) of first sensor 8a. The detection signal shown in (b) of FIG. 5 is output from one of sensing elements (e.g., sensing element CH1) of second sensor 8b. The detection signal shown in (c) of FIG. 5 is output from one of sensing elements (e.g., sensing element CH1) of third sensor 8c.

As shown in (a) of FIG. 5, the value of the detection signal (i.e., the signal intensity) output from first sensor 8a changes with time in period Tm. Specifically, in first period T1 in which first sensor 8a is exposed to the sample gas, sensing section 48 of first sensor 8a adsorbs the odorant contained in the sample gas, which increases the value of the detection signal output from first sensor 8a. After that, in second period T2 in which first sensor 8a is exposed to the nitrogen gas, sensing section 48 of first sensor 8a desorbs the odorant, which decreases the value of the detection signal output from first sensor 8a.

As shown in (b) of FIG. 5, the value of the detection signal output from second sensor 8b changes with time in period Tm. Specifically, in first period T1 in which second sensor 8b is exposed to the sample gas, the sensing section of second sensor 8b adsorbs the odorant contained in the sample gas, which increases the value of the detection signal output from second sensor 8b. At this time, as shown in (a) and (b) of FIG. 5, predetermined phase difference ΔT occurs between the detection signal output from first sensor 8a in first period T1 and the detection signal output from second sensor 8b in first period T1. Predetermined phase difference ΔT is the phase difference (e.g., two seconds) obtained by dividing first period T1 (e.g., six seconds) by the number (e.g., three) of the sensors (i.e., first sensor 8a, second sensor 8b, and third sensor 8c).

After that, in second period T2 in which second sensor 8b is exposed to the nitrogen gas, the sensing section of second sensor 8b desorbs the odorant, which decreases the value of the detection signal output from second sensor 8b. At this time, like the case described above, predetermined phase difference ΔT occurs between the detection signal output from first sensor 8a in second period T2 and the detection signal output from second sensor 8b in second period T2.

As shown in (c) of FIG. 5, the value of the detection signal output from third sensor 8c changes with time in period Tm. Specifically, in first period T1 in which third sensor 8c is exposed to the sample gas, the sensing section of third sensor 8c adsorbs the odorant contained in the sample gas, which increases the value of the detection signal output from third sensor 8c. At this time, as shown in (b) and (c) of FIG. 5, predetermined phase difference ΔT occurs between the detection signal output from second sensor 8b in first period T1 the detection signal output from third sensor 8c in first period T1.

After that, in second period T2 in which third sensor 8c is exposed to the nitrogen gas, the sensing section of third sensor 8c desorbs the odorant, which decreases the value of the detection signal output from third sensor 8c. At this time, like the case described above, predetermined phase difference ΔT occurs between the detection signal output from second sensor 8b in second period T2 and the detection signal output from third sensor 8c in second period T2.

While first sensor 8a, second sensor 8b, and third sensor 8c are the electrically resistant sensors in this embodiment, the type is not limited thereto. Examples of the sensors may include various sensors such as electrochemical sensors, semiconductor sensors, field-effect transistor sensors, surface acoustic wave sensors, and quartz crystal sensors.

Obtainer 10 obtains the detection signal output from first sensor 8a in first period T1, the detection signal output from second sensor 8b in first period T1, and the detection signal output from third sensor 8c in first period T1. Obtainer 10 then outputs the obtained detection signals to measurer 12.

Note that obtainer 10 may obtain the detection signal output from first sensor 8a in second period T2, the detection signal output from second sensor 8b in second period T2, and the detection signal output from third sensor 8c in second period T2.

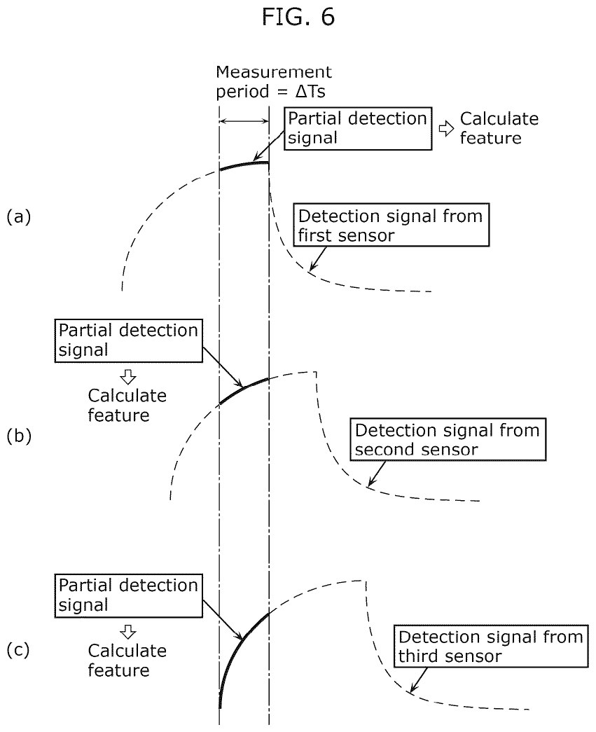

Measurer 12 measures a plurality of partial detection signals (examples of the plurality of detection signals) output in the common measurement period, which is shorter than first period T1, out of the plurality of detection signals obtained from obtainer 10. Measurer 12 outputs the measured partial detection signals to identifier 16. Note that the common measurement period is for measuring the detection signals obtained from first sensor 8a, second sensor 8b, and third sensor 8c at the same time. Here, measurement processing by measurer 12 will be described in detail with reference to FIG. 6. FIG. 6 is for illustrating an example of the measurement processing by measurer 12 of odor identification system 2 according to Embodiment 1.

As shown in (a) of FIG. 6, measurer 12 measures the partial detection signals output in common measurement period ΔTs (e.g., two seconds), which is shorter than first period T1, out of the detection signals obtained from first sensor 8a. As shown in (b) of FIG. 6, measurer 12 measures the partial detection signals output in common measurement period ΔTs, which is shorter than first period T1, out of the detection signals obtained from second sensor 8b. As shown in (c) of FIG. 6, measurer 12 measures the partial detection signals output in common measurement period ΔTs, which is shorter than first period T1, out of the detection signals obtained from third sensor 8c.

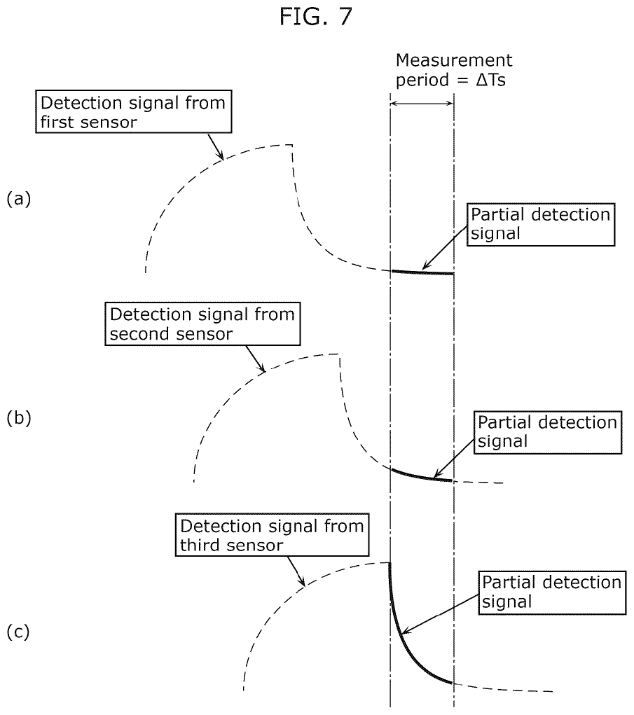

Another example of the measurement processing by measurer 12 will be described with reference to FIG. 7. FIG. 7 illustrates the other example of the measurement processing by measurer 12 of odor identification system 2 according to Embodiment 1. In the example shown in FIG. 7, obtainer 10 obtains the detection signal output from first sensor 8a in second period T2, the detection signal output from second sensor 8b in second period T2, and the detection signal output from third sensor 8c in second period T2.

As shown in (a) of FIG. 7, measurer 12 measures the partial detection signals output in common measurement period ΔTs (e.g., six seconds), which is shorter than second period T2, out of the detection signals obtained from first sensor 8a. As shown in (b) of FIG. 7, measurer 12 measures the partial detection signals output in common measurement period ΔTs, which is shorter than second period T2, out of the detection signals obtained from second sensor 8b. As shown in (c) of FIG. 7, measurer 12 measures the partial detection signals output in common measurement period ΔTs, which is shorter than second period T2, out of the detection signals obtained from third sensor 8c.

Storage 14 is a memory that stores the trained model used by identifier 16. The trained model is a logical model for identifying the sample gas. Specifically, for example, the trained model is a logical model for identifying which kind of odorant is contained in the sample gas. For example, the trained model receives, as an input, the respective features of the plurality of partial detection signals measured by measurer 12, and outputs the information indicating which kind of odorant is contained in the sample gas.

For example, the trained model is built by machine learning using a known odorant and the following features as training data. The features are of the respective partial detection signals output from first sensor 8a, second sensor 8b, and third sensor 8c exposed to the sample gas containing the known odorant. For example, a neural network, a random forest, a support vector machine, a self-organizing map, or any other suitable algorithm is used for building a logical model in machine learning.

Identifier 16 calculates the respective features of the plurality of partial detection signals measured by measurer 12. Specifically, as shown in FIG. 6, identifier 16 calculates, as a feature, the maximum value of each partial detection signal (i.e., the sensitivity of each signal), or the slope of each partial detection signal (i.e., the amount of change in the value of each partial detection signal per unit time). Identifier 16 then identifies the odor of the sample gas based on the calculated features, using the trained model stored in storage 14. Specifically, identifier 16 identifies which kind of odorant is contained in the sample gas, using the trained model. Identifier 16 outputs the information indicating a result of identification to a display (not shown) of odor identification system 2, for example. Accordingly, the display displays the result of identification by identifier 16.

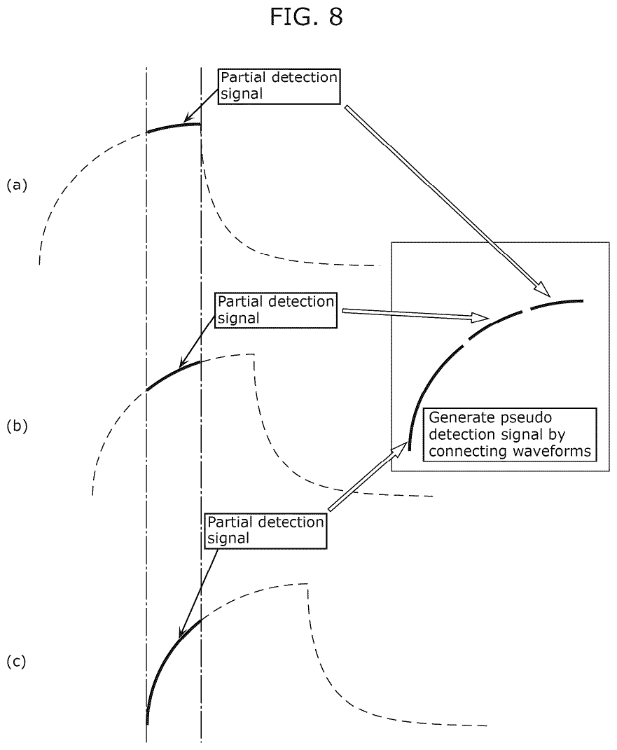

An example of identification processing by identifier 16 will be described with reference to FIG. 8. FIG. 8 illustrates the example of the identification processing by identifier 16 of odor identification system 2 according to Embodiment 1. As shown in FIG. 8, identifier 16 generates a pseudo detection signal by connecting the respective waveforms of the measured partial detection signals. A pseudo detection signal is obtained by generating, in a pseudo manner, the respective detection signals detected by first sensor 8a, second sensor 8b, and third sensor 8c during first period T1. Identifier 16 may identify the odor of the sample gas based on the generated pseudo detection signal, using the trained model stored in storage 14.

### 1-2. Operation of Odor Identification System

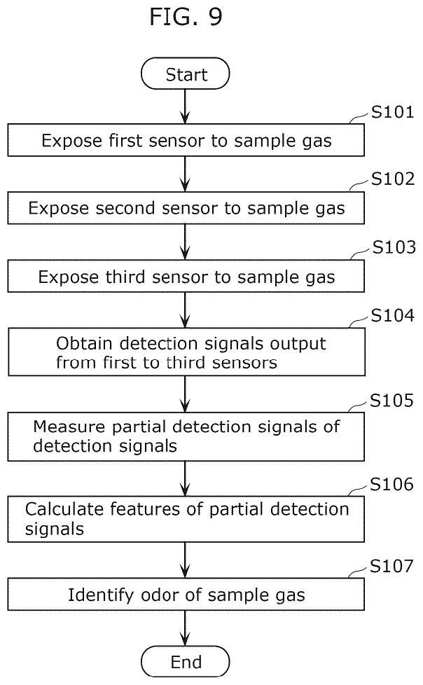

Now, an operation of odor identification system 2 according to Embodiment 1 will be described with reference to FIG. 9. FIG. 9 is a flowchart showing the flow of the operation of odor identification system 2 according to Embodiment 1.

As shown in FIG. 9, first, controller 6 drives pump 22 and switches first selector valve 20a to the first state in first period T1 out of period Tm. Accordingly, the sample gas is introduced into first container 18a and first sensor 8a is exposed to the sample gas during first period T1 out of period Tm (S101).

Next, at a lapse of the time corresponding to predetermined phase difference ΔT after switching first selector valve 20a to the first state, controller 6 drives pump 22 and switches second selector valve 20b to the first state in first period T1 out of period Tm. Accordingly, the sample gas is introduced into second container 18b and second sensor 8b is exposed to the sample gas during first period T1 out of period Tm (S102).

Then, at a lapse of the time corresponding to predetermined phase difference ΔT after switching second selector valve 20b to the first state, controller 6 drives pump 22 and switches third selector valve 20c to the first state in first period T1 out of period Tm. Accordingly, the sample gas is introduced into third container 18c, and third sensor 8c is exposed to the sample gas during first period T1 out of period Tm (S103).

After that, obtainer 10 obtains the detection signal output from first sensor 8a in first period T1, the detection signal output from second sensor 8b in first period T1, and the detection signal output from third sensor 8c in first period T1 (S104). Obtainer 10 outputs the obtained detection signals to measurer 12.

Next, measurer 12 measures the plurality of partial detection signals output in common measurement period ΔTs, which is shorter than first period T1, out of the obtained detection signals (S105). Measurer 12 outputs the measured partial detection signals to identifier 16.

Then, identifier 16 calculates the respective features of the plurality of partial detection signals measured by measurer 12 (S106), and identifies the odor of the sample gas based on the calculated features, using the trained model stored in storage 14 (S107). After that, the processing shown in the flowchart of FIG. 9 ends.

### 1-3. Advantages

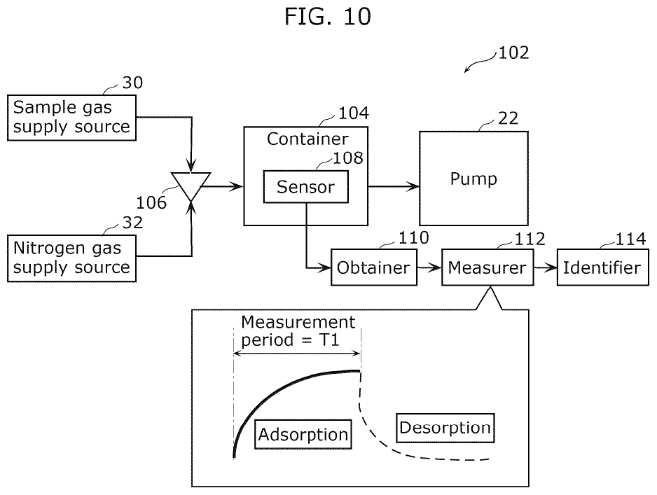

A configuration of odor identification system 102 according to Comparative Example A will be described with reference to FIG. 10. FIG. 10 illustrates the configuration of odor identification system 102 according to Comparative Example A. In FIG. 10, the same reference signs represent substantially the same configurations as in Embodiment 1 described above, and redundant description will be omitted.

As shown in FIG. 10, odor identification system 102 according to Comparative Example A includes container 104, selector valve 106, pump 22, obtainer 110, measurer 112, and identifier 114. Container 104 contains one sensor 108.

Obtainer 110 obtains the detection signal output from sensor 108 in first period t1, and outputs the obtained detection signal to measurer 112. Measurer 112 measures the obtained detection signal during the measurement period corresponding to first period T1 and outputs a result of measurement to identifier 114. Accordingly, identifier 114 identifies the odor of the sample gas, based on the detection signal output from sensor 108 during first period T1.

In the configuration of Comparative Example A has however the following problem. Since the measurement of the detection signal requires the time (e.g., six seconds) corresponding to first period T1, the odor of the sample gas cannot be identified in a short time. It is thus difficult to perform full inspection in real time as to whether food transported one by one on a conveyor causes gas with an unusual odor, if odor identification system 102 according to Comparative Example A is mounted on a food production line, for example.

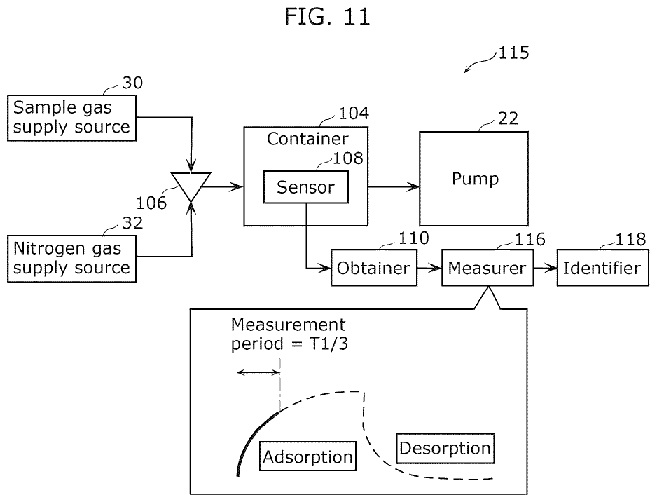

Now, a configuration of odor identification system 115 according to Comparative Example B will be described with reference to FIG. 11. FIG. 11 illustrates the configuration of odor identification system 115 according to Comparative Example B. In FIG. 11, the same reference signs represent substantially the same configurations as in Embodiment 1 and Comparative Example A described above and redundant description will be omitted.

As shown in FIG. 11, odor identification system 115 according to Comparative Example B includes container 104, selector valve 106, pump 22, obtainer 110, measurer 116, and identifier 118.

Obtainer 110 obtains the detection signal output from sensor 108 in first period T1, and outputs the obtained detection signal to measurer 116. Measurer 116 measures the obtained detection signal during the measurement period corresponding to first period T1×⅓, and outputs a result of measurement to identifier 118. Accordingly, identifier 118 identifies the odor of the sample gas, based on the detection signal output from sensor 108 during first period T1×⅓.

In the configuration of Comparative Example B has however the following problem. The time for measuring the detection signal is equal to the time (e.g., two seconds) corresponding to first period T1×⅓, which is shorter than in Comparative Example A described above. However, a smaller amount of information is obtained through the measurement by measurer 116 in the measurement period. Accordingly, the odor of the sample gas cannot be identified with high accuracy.

By contrast, in odor identification system 2 according to Embodiment 1, measurer 12 measures the plurality of partial detection signals output in common measurement period ΔTs, which is shorter than first period T1, out of the obtained detection signals. Identifier 16 then calculates the respective features of the plurality of partial detection signals measured by measurer 12, and identifies the odor of the sample gas based on the calculated features, using the trained model stored in storage 14.

Since measurement period ΔTs for measuring the plurality of detection signals is shorter than first period T1, the time required for the measurement can be shortened, and the odor of the sample gas can be identified in the short time. Since measurer 12 measures the plurality of partial detection signals in common measurement period ΔTs, a larger amount of information can be obtained through the measurement. As a result, the odor of the sample gas can be identified with high accuracy.

### 1-4. Examples and Comparative Examples

In order to confirm the above described advantages of identifying the odor of the sample gas with high accuracy, following Experiment 1 is conducted. In this Experiment 1, sample gases A, B, and C (A to C) with three types of reference odors for odor determination (odor intensity level 2) were used as sample gases.
Sample gases A to C each include a different one of the three types of chemical compounds below.
Sample gas A: β-phenylethyl alcohol (smell of flower at a concentration of 1.659 ppm)
Sample gas B: methyl cyclopentenolone (smell of something sweet and burnt at a concentration of 2.033 ppm)
Sample gas C: isovaleric acid (odor of sweaty socks at a concentration of 4.749 ppm)

As Comparative Example 1, one electrically resistant sensor including 16 sensing elements is used and exposed to a sample gases A to C. With respect to sample gases A to C, 80 pulses of the detection signals output from the sensing elements of the sensor were obtained. The detection signals output during the measurement period corresponding to first period T1 at the time of adsorption were measured. With the features extracted from the measured detection signals input to the trained model, a test for identifying the odors of the sample gases was conducted. Note that the random forest was used as the trained model.

As Comparative Example 2, one electrically resistant sensor including 16 sensing elements is used and exposed to a sample gases A to C. With respect to sample gases A to C, 80 pulses of the detection signals output from the sensing elements of the sensor were obtained. The detection signals output during the measurement period corresponding to first period T1×⅓ at the time of adsorption were measured. With the features extracted from the measured detection signals input to the trained model, a test for identifying the odors of the sample gases was conducted. Note that the random forest was used as the trained model.

As Example 1, three electrically resistant sensors each including 16 sensing elements are used and exposed to a sample gases A to C. With respect to sample gases A to C, 80 pulses of the detection signals output from the sensing elements of the three sensors were obtained. As in Embodiment 1 described above, the partial detection signals of the detection signals output from the sensing elements of the three sensors during measurement period ΔTs, which is shorter than first period T1, at the time of adsorption were measured. With the features extracted from the measured partial detection signals input to the trained model, a test for identifying the odors of the sample gases was conducted. Note that the random forest was used as the trained model.

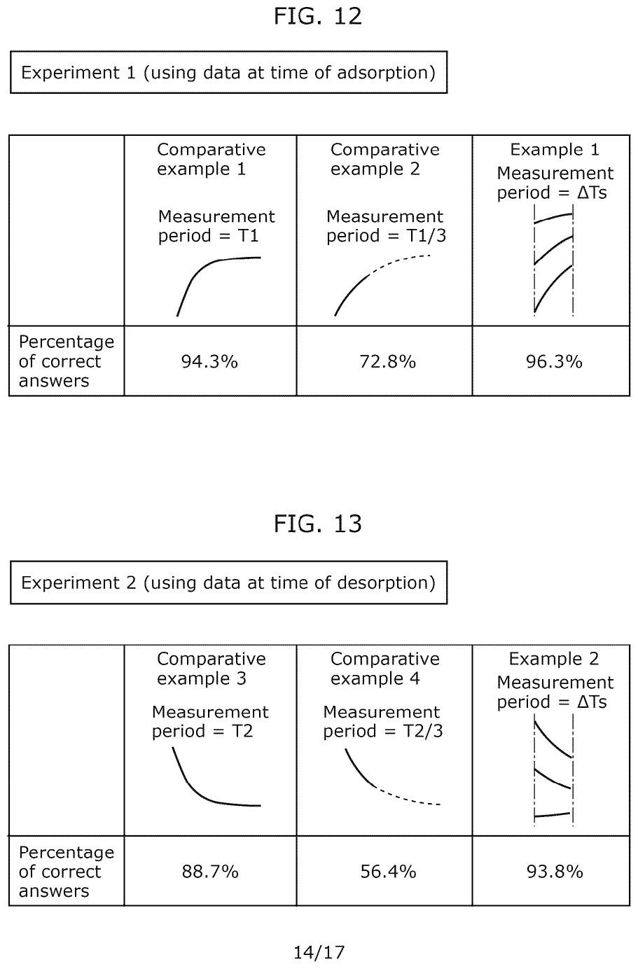

A result of Experiment 1 is as shown in FIG. 12. FIG. 12 is a table showing the result of Experiment 1 for confirming the advantages provided by odor identification system 2 according to Embodiment 1. As shown in FIG. 12, the percentages of correct answers given by the trained models in Experiment 1 were: 94.3% in Comparative Example 1, 72.8% in Comparative Example 2, and 96.3% in Example 1. In can be confirmed from the foregoing that trained model in Example 1 provides the same level of the correct answers as in Comparative Example 1, and a much higher level of the correct answers than in Comparative Example 2.

In order to further confirm the advantages described above, following Experiment 2 was conducted. In this Experiment 2, sample gases A to C were used as sample gases as in Experiment 1 described above.

As Comparative Example 3, one electrically resistant sensor including 16 sensing elements is used and exposed to a sample gases A to C. With respect to sample gases A to C, 80 pulses of the detection signals output from the sensing elements of the sensor were obtained. The detection signals output during the measurement period corresponding to second period T2 at the time of desorption were measured. With the features extracted from the measured detection signals input to the trained model, a test for identifying the odors of the sample gases was conducted. Note that the random forest was used as the trained model.

As Comparative Example 4, one electrically resistant sensor including 16 sensing elements is used and exposed to a sample gases A to C. With respect to sample gases A to C, 80 pulses of the detection signals output from the sensing elements of the sensor were obtained. The detection signals output during the measurement period corresponding to second period T2×⅓at the time of desorption were measured. With the features extracted from the measured detection signals input to the trained model, a test for identifying the odors of the sample gases was conducted. Note that the random forest was used as the trained model.

As Example 2, three electrically resistant sensors each including 16 sensing elements are used and exposed to a sample gases A to C. With respect to sample gases A to C, 80 pulses of the detection signals output from the sensing elements of the three sensors were obtained. As in Embodiment 1 described above, the partial detection signals of the detection signals output from the sensing elements of the three sensors during measurement period ΔTs, which is shorter than second period T2, at the time of desorption were measured. With the features extracted from the measured partial detection signals input to the trained model, a test for identifying the odors of the sample gases was conducted. Note that the random forest was used as the trained model.

A result of Experiment 2 is as shown in FIG. 13. FIG. 13 is a table showing the result of Experiment 2 for confirming the advantages provided by odor identification system 2 according to Embodiment 1. As shown in FIG. 13, the percentages of correct answers given by the trained models in Experiment 2 were: 88.7% in Comparative Example 3, 56.4% in Comparative Example 4, and 93.8% in Example 2. In can be confirmed from the foregoing that trained model in Example 2 provides the same level of the correct answers as in Comparative Example 3, and a much higher level of the correct answers than in Comparative Example4.

### Embodiment 2

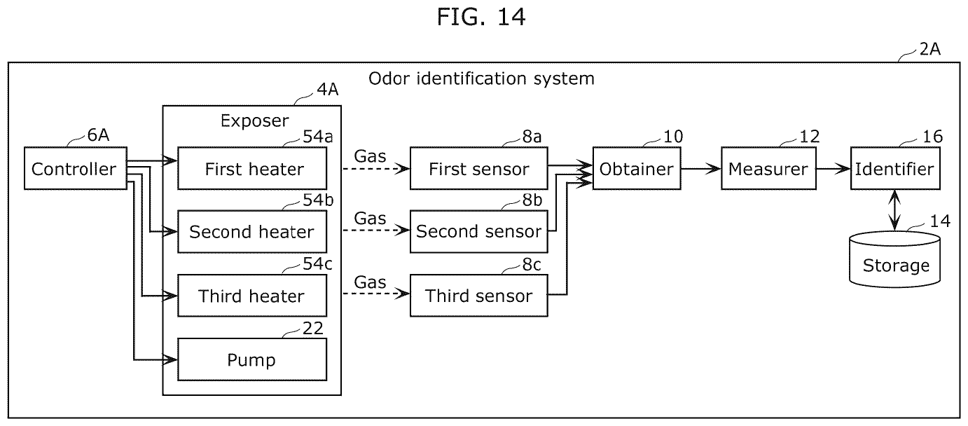

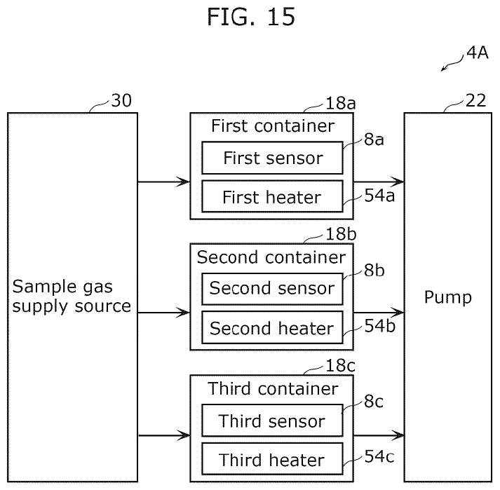

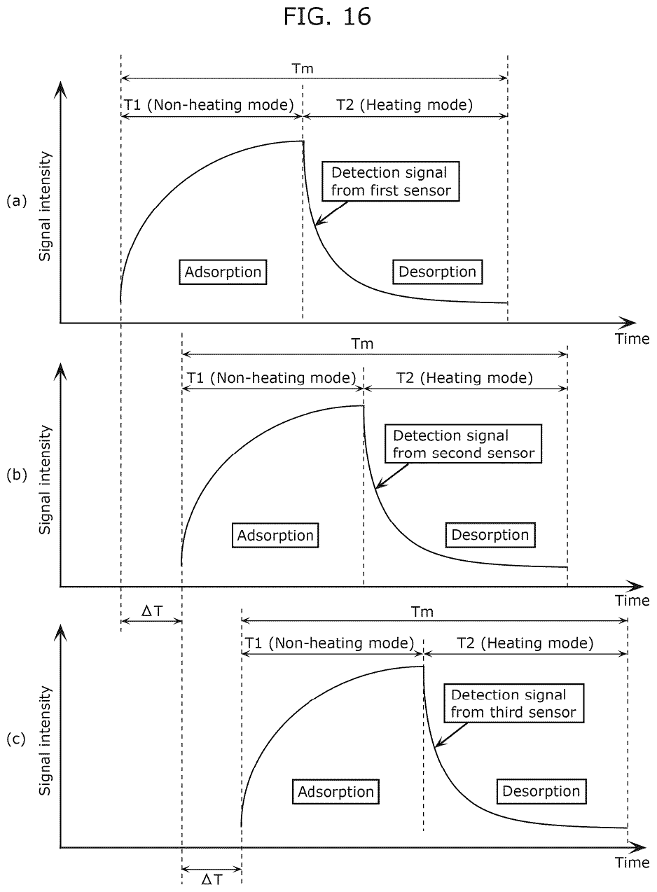

A configuration of odor identification system 2A according to Embodiment 2 will be described with reference to FIGS. 14 to 16. FIG. 14 is a block diagram showing the configuration of odor identification system 2A according to Embodiment 2. FIG. 15 is a schematic view showing exposer 4A of odor identification system 2A according to Embodiment 2. FIG. 16 is a graph schematically showing the respective detection signals output from first sensor 8a, second sensor 8b, and third sensor 8c. In this embodiment, the same reference signs represent substantially the same configurations as in Embodiment 1 described above, and redundant description will be omitted.

As shown in FIG. 14, in odor identification system 2A according to Embodiment 2, exposer 4A includes first heater 54a (as an example of the temperature control device), second heater 54b (as another example of the temperature control device), and third heater 54c (as further another example of the temperature control device) in place of first selector valve 20a, second selector valve 20b, and third selector valve 20c described above in Embodiment 1. First heater 54a, second heater 54b, and third heater 54c are each, for example, an electric heater that generates heat by means of the electrical resistance to the supplied electricity, and is controlled by controller 6A.

As shown in FIG. 15, first heater 54a is contained inside first container 18a in contact with first sensor 8a. Note that the sample gas supplied from sample gas supply source 30 is introduced into first container 18a. First heater 54a alternates the heating and non-heating of first sensor 8a exposed to the sample gas in the cycle corresponding to period Tm. Specifically, as shown in (a) of FIG. 16, first heater 54a alternates: a non-heating mode not heating first sensor 8a in first period T1 of period Tm; and a heating mode of heating first sensor 8a in second period T2 of period Tm. In the heating mode, first heater 54a heats first sensor 8a so as to raise the temperature of first sensor 8a. In the non-heating mode, first heater 54a stops heating first sensor 8a, which then releases the heat so as to have a lower temperature.

In the non-heating mode, first sensor 8a adsorbs the odorant contained in the sample gas. After that, in the heating mode, first sensor 8a is heated by first heater 54a and desorbs (volatilizes) the odorant adsorbed by first sensor 8a itself in the non-heating mode right before the heating mode so as to be cleaned.

As shown in FIG. 15, second heater 54b is contained inside second container 18b in contact with second sensor 8b. Note that the sample gas supplied from sample gas supply source 30 is introduced into second container 18b. Second heater 54b alternates the heating and non-heating of second sensor 8b exposed to the sample gas in the cycle corresponding to period Tm. Specifically, as shown in (b) of FIG. 16, second heater 54b alternates: a non-heating mode not heating second sensor 8b in first period T1 of period Tm; and a heating mode of heating second sensor 8b in second period T2 of period Tm. In the heating mode, second heater 54b heats second sensor 8b so as to raise the temperature of second sensor 8b. In the non-heating mode, second heater 54b stops heating second sensor 8b, which then releases the heat so as to have a lower temperature.

In the non-heating mode, second sensor 8b adsorbs the odorant contained in the sample gas. After that, in the heating mode, second sensor 8b is heated by second heater 54b and desorbs (volatilizes) the odorant adsorbed by second sensor 8b itself in the non-heating mode right before the heating mode so as to be cleaned.

As shown in FIG. 15, third heater 54c is contained inside third container 18c in contact with third sensor 8c. Note that the sample gas supplied from sample gas supply source 30 is introduced into third container 18c. Third heater 54c alternates the heating and non-heating of third sensor 8c exposed to the sample gas in the cycle corresponding to period Tm. Specifically, as shown in (c) of FIG. 16, third heater 54c alternates: a non-heating mode not heating third sensor 8c in first period T1 of period Tm; and a heating mode of heating third sensor 8c in second period T2 of period Tm. In the heating mode, third heater 54c heats third sensor 8c so as to raise the temperature of third sensor 8c. In the non-heating mode, third heater 54c stops heating third sensor 8c, which then releases the heat so as to have a lower temperature.

In the non-heating mode, third sensor 8c adsorbs the odorant contained in the sample gas. After that, in the heating mode, third sensor 8c is heated by third heater 54c and desorbs (volatilizes) the odorant adsorbed by third sensor 8c itself in the non-heating mode right before the heating mode so as to be cleaned.

This embodiment provides at least the same advantages as in Embodiment 1 described above.

### Other Variations

While the odor identification system according to one or more aspects has been described above based on the embodiments, the present disclosure is not limited to the embodiments. The present disclosure may include forms obtained by various modifications to the foregoing embodiments that can be conceived by those skilled in the art or forms achieved by freely combining the elements and functions in the foregoing embodiments without departing from the scope and spirit of the present disclosure.

While being three (i.e., first sensor 8a, second sensor 8b, and third sensor 8c) in the embodiments described above, the number of the sensors is not limited thereto. For example, the number may be any number such as two, four, or more.

While first selector valve 20a, second selector valve 20b, and third selector valve 20c are electromagnetic valves in Embodiment 1 described above, the type is not limited thereto. The selector valves may be pressure valves, for example.

While obtainer 10 directly obtains the respective detection signals output from first sensor 8a, second sensor 8b, and third sensor 8c in the embodiments described above, the configuration is not limited thereto. For example, obtainer 10 may obtain the respective detection signals output from first sensor 8a, second sensor 8b, and third sensor 8c via a network. In this case, first sensor 8a, second sensor 8b, and third sensor 8c may be located outside odor identification system 2 (or 2A).

In the embodiment described above, the elements may be achieved by dedicated hardware or a program executor, such as a CPU or a processor, which reads out software programs stored in a recording medium, such as a hard disk or a semiconductor memory, and executes the read-out programs.

A part or all of the elements the configuration of the odor identification system according to the present disclosure may be directed to achieved by dedicated hardware or a program executor, such as a CPU, executing programs.

A part or all of the elements of the above-described devices may be configured as an IC card which can be attached to and detached from the respective devices or as a stand-alone module. The IC card or the module is a computer system configured from a microprocessor, a ROM, and a RAM, for example. The IC card or the module may include a super-multifunctional LSI. The IC card or the module achieves its function through the microprocessor's operation according to the computer program. The IC card or the module may be tamper-resistant.

The present disclosure may be implemented as the above-described method. Moreover, the present disclosure may be directed to a computer program for executing the above-described method using a computer, or may be a digital signal generated by the computer program. Furthermore, the present disclosure may also be implemented as a non-transitory computer-readable recording medium, such as a flexible disk, hard disk, CD-ROM, MO, DVD, DVD-ROM, DVD-RAM, Blu-ray (registered trademark) disc (BD), semiconductor memory, or any other suitable means, having recorded thereon the computer program or the digital signal. Furthermore, the present disclosure may include the digital signal recorded on the recording medium. Furthermore, the present disclosure may be implemented by transmitting the computer program or the digital signal via, for example, an electric communication line, a wireless or wired communication line, a network such as the Internet, or data broadcasting. Furthermore, the present disclosure may be directed to a computer system including a memory storing the computer program and a microprocessor that operates according to the computer program. Furthermore, the present disclosure may be implemented by another independent computer system by transmitting, to the independent computer system, the program or the digital signal recorded on the recording medium, or by transmitting, to the independent computer system, the program or the digital signal via the network or any other suitable means.

### INDUSTRIAL APPLICABILITY

The odor identification system according to the present disclosure is useful as a system, for example, for inspecting whether food in a food production line has an unusual odor.

### REFERENCE SIGNS LIST

2, 2A, 102, 115 odor identification system
4, 4A exposer
6, 6A controller
8a first sensor
8b second sensor
8c third sensor
10, 110 obtainer
12, 112, 116 measurer
14 storage
16, 114, 118 identifier
18a first container
18b second container
18c third container
20a first selector valve
20b second selector valve
20c third selector valve
22 pump
24, 34, 40 first inlet port
26, 36, 42 second inlet port
28, 38, 44 outlet port
30 sample gas supply source
32 nitrogen gas supply source
46 sensing element
48 sensing section
50, 52 electrode
54c third heater
104 container
106 selector valve
108 sensor

1. An odor identification method of identifying an odor of a sample gas, the odor identification method comprising:
(a) causing a plurality of sensors to adsorb an odorant contained in the sample gas at different times in a first period, and to desorb the odorant at different times in a second period, the plurality of sensors outputting respective detection signals according to an adsorption concentration of the odorant contained in the sample gas;
(b) measuring the respective detection signals output from the plurality of sensors in a common measurement period that is shorter than the first period or the second period; and
(c) identifying the odor of the sample gas, based on the respective detection signals measured in the (b) measuring.

2. The odor identification method according to claim 1, wherein
the plurality of sensors are contained in respective containers,
a plurality of selector valves are provided in association with the respective containers, the plurality of selector valves each being switched between a first state and a second state, the sample gas being introduced into the respective containers in the first state, the sample gas is discharged from the respective containers in the second state, and
the (a) causing includes switching the plurality of selector valves from one of the first state or the second state to another at different times.

3. The odor identification method according to claim 1, wherein
a plurality of temperature control devices are provided in association with the plurality of sensors that alternate a heating mode and a non-heating mode, the plurality of sensors being heated in the heating mode, the plurality of sensors being not heated in the non-heating mode, and
the (a) causing includes switching the plurality of temperature control devices from one of the heating mode or the non-heating mode to another of the heating mode or the non-heating at different times.

4. The odor identification method according to claim 1, wherein the (a) causing includes:
causing the plurality of sensors to adsorb the odorant at different times in the first period to cause the respective detection signals output from the plurality of sensors to have a predetermined phase difference; and
causing the plurality of sensors to desorb the odorant at different times in the second period to cause the respective detection signals output from the plurality of sensors to have the predetermined phase difference.

5. The odor identification method according to claim 4, wherein
the predetermined phase difference is obtained by dividing the first period or the second period by a total number of the plurality of sensors.

6. The odor identification method according to claim 1, wherein
the (c) identifying includes generating a pseudo detection signal by connecting waveforms of the respective detection signals measured in the (b) measuring, and identifying the odor of the sample gas based on the pseudo detection signal generated, using a trained model for identifying the sample gas.

7. The odor identification method according to claim 1, wherein
the (c) identifying includes calculating respective features of the respective detection signals measured in the (b) measuring, and identifying the odor of the sample gas based on the respective features calculated, using a trained model for identifying the sample gas.

8. The odor identification method according to claim 1, further comprising:
(d) obtaining, via a network, the respective detection signals output from the plurality of sensors in the first period or the second period.

9. The odor identification method according to claim 1, wherein
each of the plurality of sensors includes a plurality of sensing elements with different sensing characteristics.

10. An odor identification system for identifying an odor of a sample gas, the odor identification system comprising:
a plurality of sensors that output respective detection signals according to an adsorption concentration of an odorant contained in the sample gas;
a measurer that measures the respective detection signals output from the plurality of sensors in a common measurement period that is shorter than a first period or a second period, when the plurality of sensors adsorb the odorant contained in the sample gas at different times in the first period, and desorb the odorant contained in the sample gas at different times in the second period; and
an identifier that identifies the odor of the sample gas, based on the respective detection signals measured by the measurer.
